(19) United States
(12) Patent Application Publication
(10) Pub. No.: US 2008/0208058 A1
(43) Pub. Date: Aug. 28, 2008

(54) ULTRASOUND DIAGNOSTIC APPARATUS

(75) Inventors: Tomohiro SABATA, Tokyo (JP); Kenichi OBA, Yokohama-shi (JP); Tadaaki KANBARA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/054,888

(22) Filed: Mar. 25, 2008

Related U.S. Application Data
(63) Continuation of application No. PCT/JP2006/308737, filed on Apr. 26, 2006.

(30) Foreign Application Priority Data
Sep. 26, 2005 (JP) 2005-278749

(51) Int. Cl. A61B 8/14

(52) U.S. Cl. 600/454

(57) ABSTRACT

The ultrasound diagnostic apparatus of the present invention includes: an ultrasound transmitting/receiving unit for receiving an echo signal; dynamic blood flow detection unit for calculating a predetermined detection value based on dynamic blood flow in a test subject based on the echo signal; a luminance value conversion unit for converting the predetermined detection value into a luminance value; a luminance value replacement unit retaining luminance value conversion information; and a comparison unit for comparing a first luminance value in one frame with a second luminance value in the frame immediately preceding the one frame, out of the luminance values outputted from the luminance value conversion unit, wherein the comparison unit is adapted to output either the first luminance value or a third luminance value which is not larger than the absolute value of the second luminance value, based on the comparison result and the luminance value replacement information.

### CROSS REFERENCE TO RELATED APPLICATION

This application is a continuation application of PCT/JP2006/308737 filed on Apr. 26, 2006 and claims benefit of Japanese Application No. 2005-278749 filed in Japan on Sep. 26, 2005, the entire contents of which are incorporated herein by this reference.

### BACKGROUND OF THE INVENTION

1. Field of the Invention

The present invention relates to an ultrasound diagnostic apparatus, particularly to an ultrasound diagnostic apparatus having a function of showing dynamic blood flow in a living body.

2. Description of the Related Art

Conventionally, there has been widely used an ultrasound diagnostic apparatus which irradiates ultrasound into a living body and receives a reflected wave of the ultrasound reflected at a tissue of the living body to obtain a tomogram of the living body. Further, in recent years, there has been also widely used an ultrasound diagnostic apparatus which has a function of displaying, besides the tomogram of a living body, for example, dynamic blood flow in a living body as a blood flow image by exploiting the Doppler effect by which the frequency of a reflected wave is shifted due to the moving velocity of a reflecting object in motion.

As such an apparatus, for example, a blood-flow velocity display apparatus proposed in Japanese Patent Publication No. 06-9556 is widely known. The blood-flow velocity display apparatus of Japanese Patent Publication No. 06-9556 is configured such that after a comparison between the magnitudes of a blood-flow velocity calculation value during a current scan and a blood-flow velocity display value during the previous scan is performed by blood-flow velocity display value calculation unit, a blood-flow velocity display value is calculated based on the aforementioned comparison result and is used to display a blood flow image on a color monitor. Thus, using the blood-flow velocity display apparatus of Japanese Patent Publication No. 06-9556 enables an operator or the like to easily recognize a peak value of a pulsating blood flow velocity.

### SUMMARY OF THE INVENTION

The ultrasound diagnostic apparatus of a first aspect of the present invention includes: ultrasound transmitting/receiving unit for transmitting an ultrasound pulse to generate ultrasound to be transmitted from ultrasound transducing unit to a test subject, and receiving an echo signal outputted from the ultrasound transducing unit based on the ultrasound reflected in the test subject; based on the echo signal, dynamic blood flow detection unit for calculating a predetermined detection value based on dynamic blood flow in the test subject; luminance value conversion unit for converting the predetermined detection value into a luminance value; luminance value replacement unit retaining luminance value conversion information for converting one luminance value to another luminance value; and comparison unit for comparing a first luminance value in one frame with a second luminance value in the frame immediately preceding the one frame, out of luminance values outputted from the luminance value conversion unit, wherein the comparison unit is adapted to output either the first luminance value or a third luminance value, which is not larger than the absolute value of the second luminance value, based on the comparison result and the luminance value replacement information.

The ultrasound diagnostic apparatus of a second aspect of the present invention is the ultrasound diagnostic apparatus according to the first aspect of the present invention, wherein the comparison unit is adapted to output the first luminance value when obtaining a comparison result that the first luminance value is larger than the second luminance value, and to replace the second luminance value with the third luminance value based on the luminance value replacement information, thereby outputting the third luminance value when obtaining a comparison result that the first luminance value is not larger than the second luminance value.

The ultrasound diagnostic apparatus of a third aspect of the present invention is the ultrasound diagnostic apparatus according to the second aspect of the present invention, wherein the comparison unit is adapted to increase the attenuation of the third luminance value as the second luminance value increases when replacing the second luminance value with the third luminance value based on the luminance value replacement information.

The ultrasound diagnostic apparatus of a fourth aspect of the present invention is the ultrasound diagnostic apparatus according to the second aspect of the present invention, wherein the luminance value replacement information retained by the luminance value replacement unit is configured to be a rewritable data file.

The ultrasound diagnostic apparatus of a fifth aspect of the present invention is the ultrasound diagnostic apparatus according to the third aspect of the present invention, wherein the luminance value replacement information retained by the luminance value replacement unit is configured to be a rewritable data file.

The ultrasound diagnostic apparatus of a sixth aspect of the present invention is the ultrasound apparatus according to the fourth aspect of the present invention, wherein the data file is made up of a plurality of data files.

The ultrasound diagnostic apparatus of a seventh aspect of the present invention is the ultrasound apparatus according to the fifth aspect of the present invention, wherein the data file is made up of a plurality of data files.

The ultrasound diagnostic apparatus of an eighth aspect of the present invention is the ultrasound diagnostic apparatus according to the first aspect of the present invention, wherein the comparison unit is adapted to output the first luminance value when obtaining a comparison result that the first luminance value has a different sign from that of the second luminance value, or that the first luminance value has the same sign as that of the second luminance value and the absolute value of the first luminance value is larger than that of the second luminance value; and to replace the second luminance value with the third luminance value based on the luminance value replacement information and output the third luminance value, when obtaining a comparison result that the first luminance value has the same sign as that of the second luminance and the absolute value of the first luminance value is not larger than that of the second luminance value.

The ultrasound diagnostic apparatus of a ninth aspect of the present invention is the ultrasound diagnostic apparatus according to the eighth aspect of the present invention, wherein the comparison unit is adapted to increase the attenuation of the third luminance value as the second luminance value increases when replacing the second luminance value with the third luminance value based on the luminance value replacement information.

The ultrasound diagnostic apparatus of a tenth aspect of the present invention is the ultrasound diagnostic apparatus according to the eighth aspect of the present invention, wherein the luminance value replacement information retained by the luminance value replacement unit is configured to be a rewritable data file.

The ultrasound diagnostic apparatus of an eleventh aspect of the present invention is the ultrasound diagnostic apparatus according to the ninth aspect of the present invention, wherein the luminance value replacement information retained by the luminance value replacement unit is configured to be a rewritable data file.

The ultrasound diagnostic apparatus of a twelfth aspect of the present invention is the ultrasound diagnostic apparatus according to the tenth aspect of the present invention, wherein the data file is made up of a plurality of data files.

The ultrasound diagnostic apparatus of a thirteenth aspect of the present invention is the ultrasound diagnostic apparatus according to the eleventh aspect of the present invention, wherein the data file is made up of a plurality of data files.

The ultrasound diagnostic apparatus of a fourteenth aspect of the present invention is the ultrasound diagnostic apparatus according to the first aspect of the present invention, wherein the luminance value replacement information retained by the luminance value replacement unit is configured to be a rewritable data file.

The ultrasound diagnostic apparatus of a fifteenth aspect of the present invention is the ultrasound diagnostic apparatus according to the fourteenth aspect of the present invention, wherein the data file is made up of a plurality of data files.

The ultrasound diagnostic apparatus of a sixteenth aspect of the present invention includes: ultrasound transmitting/receiving unit for transmitting an ultrasound pulse to generate ultrasound to be transmitted from ultrasound transducing unit to a test subject, and receiving an echo signal outputted from the ultrasound transducing unit based on the ultrasound reflected in the test subject; based on the echo signal, dynamic blood flow detection unit for calculating a predetermined detection value based on dynamic blood flow in the test subject; luminance value conversion unit for converting the predetermined detection value into a luminance value; storage unit for storing a first luminance value in one frame and a second luminance value in the frame immediately preceding the one frame, out of the luminance values, and outputting the first luminance value and the second luminance value; luminance value replacement unit for replacing the second luminance value outputted from the storage unit with a third luminance value which is not larger than the second luminance value to output the third luminance value; and comparison unit for comparing the first luminance value outputted from the storage unit with the third luminance value outputted from the luminance value replacement unit to output one luminance value which is larger than the other luminance value.

The ultrasound diagnostic apparatus of a seventeenth aspect of the present invention is the ultrasound diagnostic apparatus according to the sixteenth aspect of the present invention, wherein the comparison unit is adapted to increase the attenuation of the third luminance value as the second luminance value increases when replacing the second luminance value with the third luminance value based on the luminance value replacement information.

The ultrasound diagnostic apparatus of an eighteenth aspect of the present invention is the ultrasound diagnostic apparatus according to the sixteenth aspect of the present invention, wherein the luminance value replacement information retained by the luminance value replacement unit is configured to be a rewritable data file.

The ultrasound diagnostic apparatus of a nineteenth aspect of the present invention is the ultrasound diagnostic apparatus according to the seventeenth aspect of the present invention, wherein the luminance value replacement information retained by the luminance value replacement unit is configured to be a rewritable data file.

The ultrasound diagnostic apparatus of a twentieth aspect of the present invention is the ultrasound diagnostic apparatus according to the eighteenth aspect of the present invention, wherein the data file is made up of a plurality of data files.

The ultrasound diagnostic apparatus of a twenty-first aspect of the present invention is the ultrasound diagnostic apparatus according to the nineteenth aspect of the present invention, wherein the data file is made up of a plurality of data files.

The ultrasound diagnostic apparatus of a twenty-second aspect of the present invention includes: ultrasound transmitting/receiving unit for transmitting an ultrasound pulse to generate ultrasound to be transmitted from ultrasound transducing unit to a test subject, and receiving an echo signal outputted from the ultrasound transducing unit based on the ultrasound reflected in the test subject; based on the echo signal, dynamic blood flow detection unit for calculating a predetermined detection value based on dynamic blood flow in the test subject; luminance value conversion unit for converting the predetermined detection value into a luminance value; and comparison unit for comparing a first luminance value in one frame with a second luminance value in the frame immediately preceding the one frame, out of the luminance values outputted from the luminance value conversion unit, wherein the ultrasound diagnostic apparatus further includes a lookup table including a comparative table for converting the second luminance value into a third luminance value which is not larger than the absolute value of the second luminance value, and wherein the comparison unit outputs either the first luminance value or the third luminance value based on the comparison result and the comparative table.

The ultrasound diagnostic apparatus of a twenty-third aspect of the present invention is the ultrasound diagnostic apparatus according to the twenty-second aspect of the present invention, wherein the comparative table retained by the lookup table is configured to be a rewritable data file.

The ultrasound diagnostic apparatus of a twenty-fourth aspect of the present invention is the ultrasound diagnostic apparatus according to the twenty-third aspect of the present invention, wherein the data file is made up of a plurality of data files.

### BRIEF DESCRIPTION OF THE DRAWINGS

FIG. 1 shows the configuration of principal parts of an ultrasound diagnostic system in which the ultrasound diagnostic apparatus relating to a present embodiment is used;

FIG. 2 shows a configuration example of an after image processing circuit included in the ultrasound diagnostic apparatus of FIG. 1;

FIG. 3 is a graph to show the relationship between the input luminance value and the output luminance value corresponding to the input luminance value in a comparative table included in the lookup table in the after image processing circuit of FIG. 2;

FIG. 4 is a graph to show the variation of the luminance value of the portion where blood flow exists, in a blood flow image displayed on a monitor;

FIG. 5 shows a configuration example of a variation of the after image processing circuit of FIG. 2; and

FIG. 6 shows another example, which is different from that of FIG. 5, of a variation of the after image processing circuit of FIG. 2.

### DETAILED DESCRIPTION OF THE PREFERRED EMBODIMENT(S)

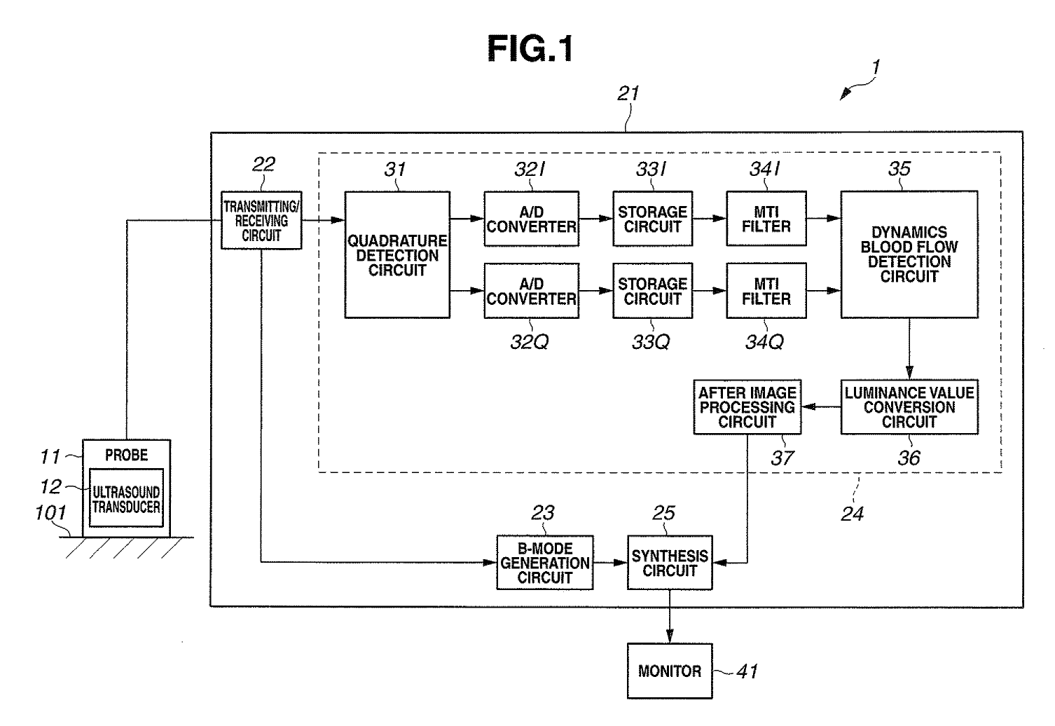

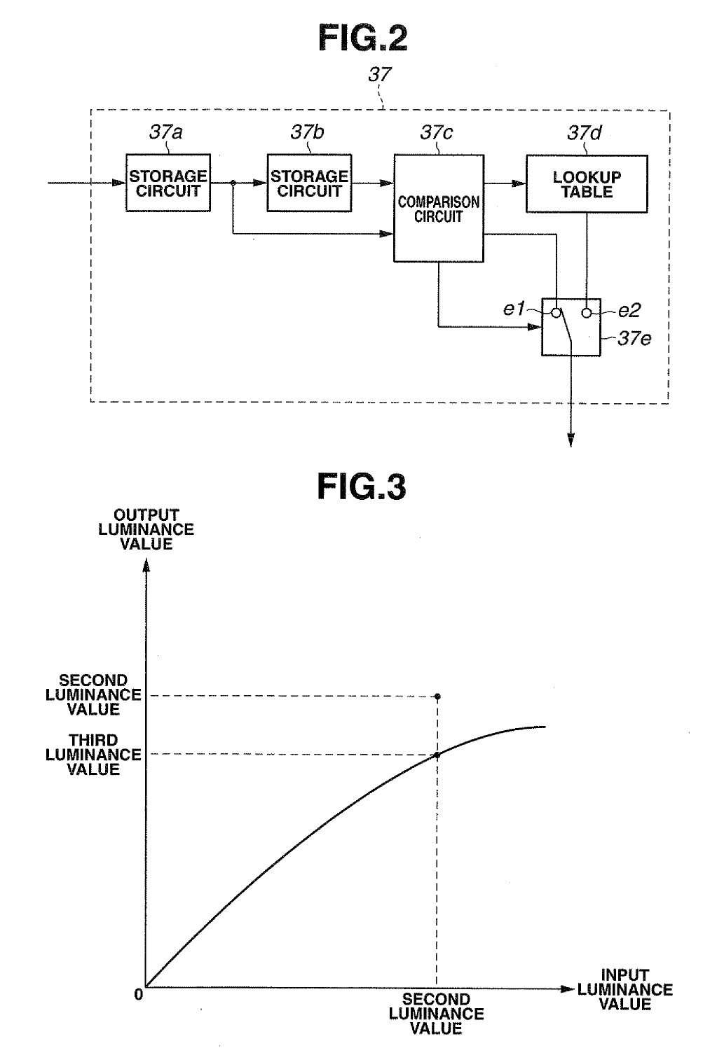

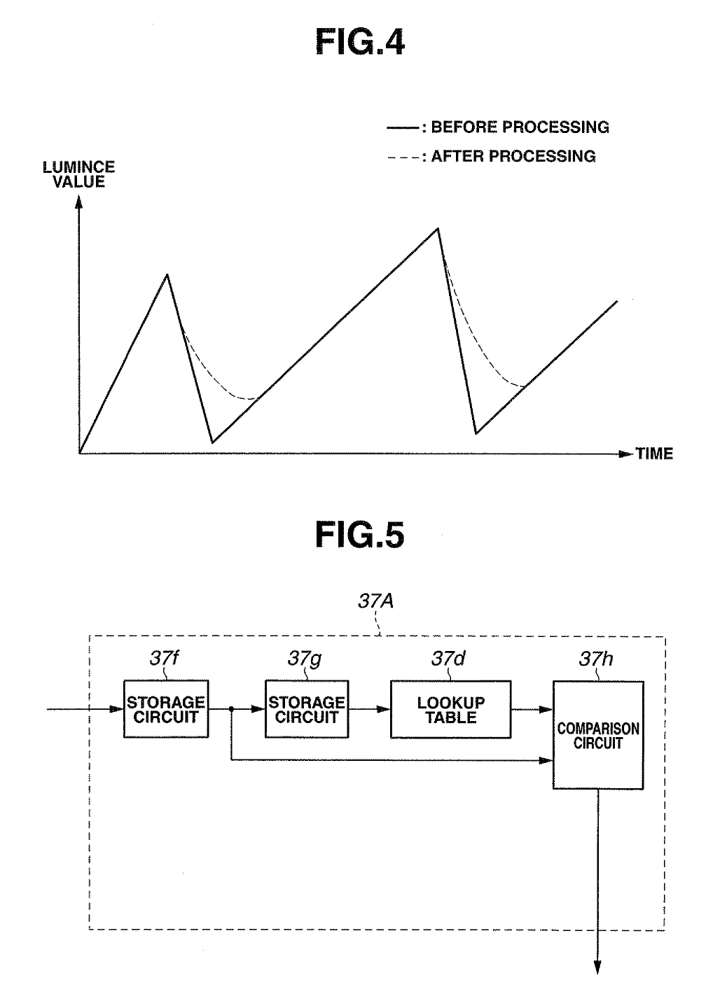

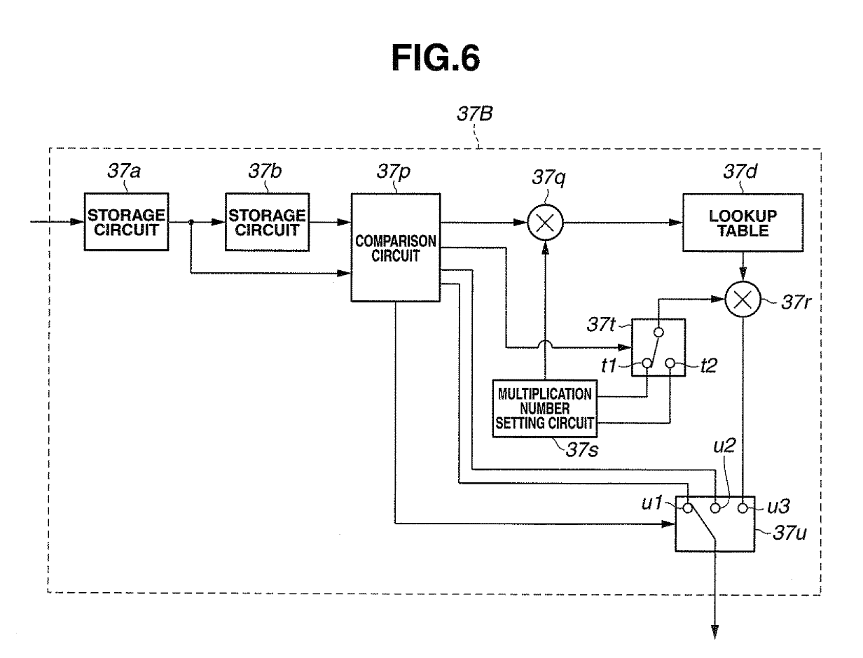

Hereinafter, embodiments of the present invention will be described with reference to the drawings. FIG. 1 shows the configuration of principal parts of an ultrasound diagnostic system in which the ultrasound diagnostic apparatus relating to a present embodiment is used. FIG. 2 shows a configuration example of an after image processing circuit included in the ultrasound diagnostic apparatus of FIG. 1. FIG. 3 is a graph to show the relationship between the input luminance value and the output luminance value corresponding to the input luminance value in a comparative table included in the lookup table in the after image processing circuit of FIG. 2. FIG. 4 is a graph to show the variation of the luminance value of the portion where blood flow exists, in a blood flow image displayed on a monitor. FIG. 5 shows a configuration example of a variation of the after image processing circuit of FIG. 2. FIG. 6 shows another example, which is different from that of FIG. 5, of a variation of the after image processing circuit of FIG. 2.

The ultrasound diagnostic system 1 is configured to include a probe 11, an ultrasound diagnostic apparatus 21, and a monitor 41.

The probe 11 contains thereinside an ultrasound transducer 12 which transmits ultrasound to a test subject 101, for example, a living body, and receives a reflected wave of the ultrasound in the test subject 101.

The ultrasound transducer 12 as the ultrasound transducing unit transmits ultrasound to the test subject 101 based on the ultrasound pulse signal from the ultrasound diagnostic apparatus 21, and receives the reflected wave of the ultrasound in the test subject 101 to output it as an echo signal to the ultrasound diagnostic apparatus 21.

Further, the ultrasound diagnostic apparatus 21 is configured to include a transmitting/receiving circuit 22, a B-mode generation circuit 23, a Doppler processing circuit 24, and a synthesis circuit 25.

The transmitting/receiving circuit 22 as ultrasound transmitting/receiving unit transmits an ultrasound pulse signal for driving the ultrasound transducer 12 to the probe 11 and receives the echo signal outputted from the ultrasound transducer 12 to perform processing thereon amplification and the like, and outputs the processed echo signal to the B-mode generation circuit 23 and the Doppler processing circuit 24.

The B-mode generation circuit 23 generates a tomogram of the test subject 101 based on the echo signal outputted from the transmitting/receiving circuit 22 and thereafter outputs the tomogram as a tomography signal to the synthesis circuit 25.

The Doppler processing circuit 24 is configured to include a quadrature detection circuit 31, A/D (analog/digital) converters 321 and 32Q, storage circuits 331 and 33Q, MTI (Moving Target Indicator) filters 341 and 34Q, a dynamic blood flow detection circuit 35, a luminance value conversion circuit 36, and an after image processing circuit 37.

The quadrature detection circuit 31 performs quadrature detection based on the echo signal outputted from the transmitting/receiving circuit 22 to output an in-phase echo signal, which is an in-phase component of the echo signal, to the A/D converter 321 and also to output a quadrature echo signal, which is a quadrature component of the echo signal, to the A/D converter 32Q.

The A/D converter 321 digitally converts an in-phase echo signal outputted from the quadrature detection circuit 31 and outputs the digitally converted in-phase echo signal to the storage circuit 331. Further, the A/D converter 32Q digitally converts a quadrature echo signal outputted from the quadrature detection circuit 31 and outputs the digitally converted quadrature echo signal to the storage circuit 33Q.

Further, configuration may be such that the echo signal outputted from the transmitting/receiving circuit 22 is converted into a digital signal by the A/D converter and thereafter is subjected to quadrature detection by a quadrature detection circuit made up of a digital filter.

The storage circuit 331 made up of a memory and the like stores, for example, only one frame of the in-phase echo signal outputted from the A/D converter 321 as the signal value which will be needed for the processing performed by the dynamic blood flow detection circuit 35, and thereafter outputs the signal value of the in-phase echo signal to the MTI filter 34I. Further, the storage circuit 33Q made up of a memory and the like stores, for example, only one frame of the quadrature echo signal outputted from the A/D converter 32Q as the signal value which will be needed for the processing performed by the dynamic blood flow detection circuit 35, and thereafter outputs the signal value of the in-phase echo signal to the MTI filter 34Q.

The MTI filter 341 performs a processing to remove a component, which moves slowly in a living body like an organ, from the in-phase echo signal outputted from the storage circuit 33I and thereafter outputs the processed in-phase echo signal to the dynamic blood flow detection circuit 35. Further, MTI filter 34Q performs a processing to remove a component which moves slowly in a living body like an organ, from the quadrature echo signal outputted from the storage circuit 33Q and thereafter outputs the processed quadrature echo signal to the dynamic blood flow detection circuit 35.

The dynamic blood flow detection circuit 35 is configured to include: a complex autocorrelation processing circuit for generating and outputting complex data representing a complex autocorrelation vector based on the in-phase echo signal and the quadrature echo signal outputted from the MTI filters 341 and 34Q; a frame memory retaining a frame of complex data; a weighted addition circuit for weighting and adding the inputted complex data; a velocity/intensity calculation circuit for calculating the velocity and the intensity of blood flow, and the amplitude of the complex data, based on the weighted and added data; and a threshold processing circuit for removing a component with a velocity not larger than a predetermined velocity and a component with am amplitude not larger than a predetermined amplitude. With the above described configuration, the dynamic blood flow detection circuit 35 can perform the processing to calculate a velocity value to indicate the velocity of blood flow in a living body or a power value to indicate the intensity of blood flow in a living body, as the predetermined detection value based on the dynamic blood flow in a living body, based on the in-phase echo signal and quadrature echo signal outputted from the MTI filters 34I and 34Q. Further, the dynamic blood flow detection circuit 35 outputs the velocity value or the power value which has been obtained through the aforementioned processing, as velocity value data or power value data to the luminance value conversion circuit 36.

The luminance value conversion circuit 36 performs processing based on the Doppler effect on the velocity value data or the power value data outputted from the dynamic blood flow detection circuit 35 to convert the velocity value data into luminance value data for each frame, and thereafter outputs the luminance value data to the after image processing circuit 37.

The after image processing circuit 37 is configured, as shown in FIG. 2, to include storage circuits 37a and 37b, a comparison circuit 37c, a lookup table 37d, and a switch 37e.

The storage circuit 37a, which is made up of a frame memory and the like, temporarily stores first luminance value data, which is luminance value data in one frame, and second luminance value data, which is luminance value data in the frame immediately preceding the one frame, out of the luminance value data outputted from the luminance value conversion circuit 36, thereafter outputting the first luminance value data to the comparison circuit 37c as well as outputting the second luminance value data to the storage circuit 37b.

The storage circuit 37b, which is made up of a frame memory and the like, temporarily stores the second luminance value data outputted from the storage circuit 37a and thereafter outputs the second luminance value data to the comparison circuit 37c.

The comparison circuit 37c compares the first luminance value based on the first luminance value data outputted from the storage circuit 37a with the second luminance value based on the second luminance value data outputted from the storage circuit 37b. Moreover, the first luminance value and the second luminance value are both supposed to be a positive whole number not less than 0, for example, 0 to 255.

The comparison circuit 37c outputs the first luminance value data to the switch 37e when, for example, the first luminance value is larger than the second luminance value. Further, the comparison circuit 37c controls the switching of the switch 37e so as to turn a terminal e1 into a conduction state so that the first luminance value data is outputted to the synthesis circuit 25.

Further, for example, when the first luminance value is not larger than the second luminance value, the comparison circuit 37c performs the processing to replace the second luminance value data having the second luminance value with third luminance value data having a third luminance value described later making reference to the lookup table 37d, and thereafter outputs the third luminance value data to the switch 37e. Further, the comparison circuit 37c controls the switching of the switch 37e so as to turn a terminal e2 into a conduction state such that the third luminance value data is outputted to the synthesis circuit 25. Moreover, the third luminance value is supposed to be a positive whole number not less than 0, for example, 0 to 255.

The lookup table 37d as luminance value replacement unit is configured to include a memory not shown, an input/output circuit, and the like. The memory not shown includes a comparative table containing luminous value conversion information which is to be referred to when the processing to replace a second luminance value based on the second luminance value data with a third luminance value which is preset as a value not larger than the absolute value of the second luminance value is performed. Further, the comparative table included in the lookup table 37d contains a plurality of combinations of an input luminance value, which is for example a whole number from 0 to 255, and an output luminance value, which is a whole number from 0 to 255, as the luminance value corresponding to the input luminance value. Further, each of the combinations of two luminance values has a relationship that the attenuation of the third luminance value, as the output luminance value, increases as the second luminance value, as the input luminance value, increases as shown as a nonlinear graph in FIG. 3.

In the present embodiment, the comparative table included in a memory not shown of the lookup table 37d is configured such that an input luminance value and the output luminance value corresponding to the input luminance value can be rewritten into a desired value, and for example can be configured to be a data file of a text data form. Further, the aforementioned data file is for example configured such that file identification data such as an identification code is written in the head of each data file, and in the rear of the portion in which the file identification data is written, for example, there are written a combination of two luminance values: an input luminance value which is a whole number from 0 to 255, and an output luminance value which corresponds to the input luminance value and is a whole number from 0 to 255.

Further, the lookup table 37d is not limited to a configuration in which the memory not shown has only one comparative table, and may be configured such that the memory not shown has a plurality of comparative tables. Further, when the memory not shown of the lookup table 37d has a plurality of comparative tables, the configuration may be such that an operator or the like can select a desired comparative table out of the plurality of comparative tables or that the comparison circuit 37c can select one comparative table depending on the comparison result.

The synthesis circuit 25 generates a blood flow image to show the dynamic state of blood flow in the test subject 101 based on the luminance value data outputted from the Doppler processing circuit 24. Then, the synthesis circuit 25 synthesizes a blood flow image generated based on the luminance value data outputted from the Doppler processing circuit 24 and a tomogram of the test subject 101 based on the tomography signal outputted from the B-mode generation circuit 23, and outputs the synthesized blood flow image and the tomogram as synthesized image data to the monitor 41.

The monitor 41 displays an image in which a tomogram of the test subject 101 as a living body is superimposed with a blood flow image based on the synthesized image signal outputted from the synthesis circuit 25.

Next, effects of the ultrasound diagnostic system 1 in the present embodiment will be described.

First, an operator or the like puts the probe 11 into contact with a desired observation site of the test subject 101 and thereafter makes the ultrasound transducer 12 oscillate by operating a switch not shown and provided on the exterior surface of the ultrasound diagnostic apparatus 21 to transmit ultrasound to the test subject 101.

After transmitting ultrasound to the test subject 101, the ultrasound transducer 12 receives the reflected wave of the ultrasound in the test subject 101 and outputs it as an echo signal to the ultrasound diagnostic apparatus 21.

The ultrasound diagnostic apparatus 21 generates a tomography signal at the B-mode generation circuit 23 based on the echo signal outputted from the ultrasound transducer 12 of the probe 11, and outputs either one of the first luminance value data or the third luminance value data at the Doppler processing circuit 24. The ultrasound diagnostic apparatus 21 synthesizes a blood flow image generated based on the luminance value data outputted from the Doppler processing circuit 24 and a tomogram of the test subject 101 based on the tomography signal outputted from the B-mode generation circuit 23, and outputs the blood flow image and the tomogram after synthesis as synthesized image data to the monitor 41.

The monitor 41 displays an image in which a tomogram of the test subject 101 as a living body is superimposed with a blood flow image to show dynamic blood flow in the test subject 101, based on the synthetic image signal outputted from the synthesis circuit 25.

With the above described series of processing being performed by the ultrasound diagnostic apparatus 21, an operator or the like can visually recognize dynamic blood flow in the test subject 101 in the blood flow image displayed on the monitor 41. Further, the blood flow image to be displayed on the monitor 41 may be shown for example as an image in which, when velocity value data is outputted from the dynamic blood flow detection circuit 35, the larger the blood flow velocity is, the brighter the red color becomes, that is, the magnitude of the blood flow velocity is related to the brightness of the red color. Further, the blood flow image to be displayed on the monitor 41 may be shown for example as an image in which when power value data is outputted from the dynamic blood flow detection circuit 35, the larger the blood flow intensity is, the brighter the red color becomes, that is, the magnitude of the blood flow intensity is related to the brightness of the red color.

Further, with the above described series of processing being performed by the ultrasound diagnostic apparatus 21, an operator or the like can visually recognize the blood flow velocity in the site where blood flow exists or dynamic blood flow while blood flow intensity is reduced, as a state in which the brightness of red color is gradually reduced as time elapses, in the blood flow image displayed on the monitor 41. Moreover, FIG. 4 is a graph to show temporal change of a luminance value in the portion where blood flow exists in the blood flow image displayed on the monitor 41, both when the above described series of processing has not be performed by the ultrasound diagnostic apparatus 21 and when the above described series of processing has been performed by the ultrasound diagnostic apparatus 21.

Moreover, in the present embodiment the after image processing circuit 37 included in the Doppler processing circuit 24 of the ultrasound diagnostic apparatus 21 may be configured as an after image processing circuit 37A having approximately same effect as the after image processing circuit 37 as shown in FIG. 5.

The after image processing circuit 37A is configured to include a lookup table 37d, storage circuits 37f and 37g, and a comparison circuit 37h.

The storage circuit 37f made up of a frame memory and the like temporarily stores first luminance value data which is luminance value data in one frame and second luminance value data which is luminance value in the frame immediately preceding the one frame, out of the luminance value data outputted from the luminance value conversion circuit 36, thereafter outputting the first luminance value data to the comparison circuit 37h as well as outputting the second luminance data to the storage circuit 37g.

The storage circuit 37g, which is made up of a frame memory and the like, temporarily stores the second luminance value data outputted from the storage circuit 37f and thereafter outputs the second luminance value data to the lookup table 37d.

The lookup table 37d replaces the second luminance value data outputted from the storage circuit 37g with third luminance value data based on the comparative table included in the memory not shown, and thereafter outputs the third luminance value data to the comparison circuit 37h.

The comparison circuit 37h compares the first luminance value data outputted from the storage circuit 37f with the third luminance value data outputted based on the comparative table included in the lookup table 37d, and based on the comparison result, outputs one luminance value data which has a larger luminance value to the synthesis circuit 25.

The after image processing circuit 37A having a configuration as described above can achieve an approximately same effect as that of the after image processing circuit 37 as described above.

Further, although in the description of the present embodiment, the above described first, second, and third luminance values have been assumed to be all positive whole numbers not less than 0, for example 0 to 255, they are not limited thereto. For example, the luminance values may be values including color data having a positive or negative sign, for example, a whole number in the range of −127 to +127. In such a case, the after image processing circuit 37 included in the Doppler processing circuit 24 of the ultrasound diagnostic apparatus 21 is configured to be for example an after image processing circuit 37B as shown in FIG. 6.

The after image processing circuit 37B is configured to include a storage circuit 37a, a storage circuit 37b, a lookup table 37d, a comparison circuit 37p, multipliers 37q and 37r, a multiplication number setting circuit 37s for outputting a preset multiplication number, a switch 37t having two terminals t1 and t2, and a switch 37u having three terminals u1, u2 and u3.

The comparison circuit 37p compares the first luminance value based on the first luminance value data outputted from the storage circuit 37a with the second luminance value based on the second luminance value data outputted from the storage circuit 37b.

And when the first luminance value has a different sign from that of the second luminance value, the comparison circuit 37p outputs the first luminance value data to the switch 37u. Further, the comparison circuit 37p controls the switching of the switch 37u so as to change the terminal u1 into a conduction state so that the first luminance value data is outputted to the synthesis circuit 25.

Further, when the first luminance value and the second luminance value have the same sign, the comparison circuit 37p calculates the absolute value of the first luminance value and the absolute value of the second luminance value, and further performs the comparison based on the calculation result.

And, for example, when the absolute value of the first luminance value is larger than that of the second luminance value, the comparison circuit 37p outputs the first luminance value data to the switch 37u. Further, the comparison circuit 37p controls the switching of the switch 37u so as to change the terminal u2 into a conduction state so that the first luminance value data is outputted to the synthesis circuit 25.

Further, for example, when the absolute value of the first luminance value is not larger than that of the second luminance value, the comparison circuit 37p outputs the absolute value of the second luminance value to the multiplier 37q to cause a processing, which will be described below, to be performed at the multiplier 37q, the lookup table 37d and the multiplier 37r, and controls the switching of the switch 37u so as to change the terminal u3 into a conduction state so that the luminance value data obtained by the aforementioned processing is outputted to the synthesis circuit 25.

Further, when the second luminance value is not less than 0, the comparison circuit 37p controls the switching of the switch 37t so as to turn the terminal t1 into a conduction state. On the other hand, when the second luminance value is less than 0, the comparison circuit 37p controls the switching of the switch 37t so as to change the terminal t2 into a conduction state.

The multiplication number setting circuit 37s outputs, for example, 2 as the first multiplication number to the multiplier 37q; for example, ½ as the second multiplication number to the terminal t1 of the switch 37t; and for example, −½ as the third multiplication number to the terminal t2 of the switch 37, based on the preset multiplication number.

The absolute value of the second luminance value outputted from the comparison circuit 37p is doubled by being subjected to processing based on the first multiplication number outputted from the multiplication number setting circuit 37s at the multiplier 37q; and thereafter is outputted to the lookup table 37d.

Then, the absolute value of the second luminance value, which has been doubled and outputted from the multiplier 37q, is replaced with a third luminance value based on the comparative table included in the lookup table 37d, and thereafter is outputted to the multiplier 37r.

For example, when the second luminance value is not less than 0, the third luminance value outputted from the lookup table 37d is multiplied by ½ by being subject to processing based on the second multiplication number outputted from the multiplication number setting circuit 37s at the multiplier 37r; and thereafter is outputted to the synthesis circuit 25 via the terminal u3 of the switch 37u. On the other hand, when the second luminance value is less than 0, the third luminance value outputted from the lookup table 37d is multiplied by −½ by being subject to processing based on the third multiplication number outputted from the multiplication number setting circuit 37s at the multiplier 37r; and thereafter is outputted to the synthesis circuit 25 via the terminal u3 of the switch 37u.

The after image processing circuit 37B configured as described above can achieve approximately same effect as that of the after image processing circuit 37 described above using the same lookup table 37d as that of the after image processing circuit 37 even when a value including color data having a positive or negative sign is inputted.

The ultrasound diagnostic apparatus 21 of the present embodiment performs the processing based on the comparative table included in the lookup table 37d when showing dynamic blood flow during the decrease of blood flow velocity in the blood flow image displayed on the monitor 41. Therefore, the ultrasound diagnostic apparatus 21 of the present embodiment does not need to perform the processing to calculate the luminance value for each frame by arithmetic operation using a relatively complex function, for example by taking the 0.6th power of the inputted luminance value, as conventionally practiced when showing dynamic blood flow during the decrease of blood flow velocity. Since it is possible to preinstall calculation results using a complex function as described above into the comparative table included in the lookup table 37d, the ultrasound diagnostic apparatus 21 of the present embodiment can show dynamic blood flow during the decrease of blood flow velocity by a simpler configuration than ever without performing the processing which is conventionally performed and requires large load, for example performing the aforementioned calculation for each frame in the calculation circuit for calculating luminance values. As a result, the ultrasound diagnostic apparatus 21 of the present embodiment can show dynamic blood flow in the blood flow image displayed in the monitor 41 without decreasing the frame rate.

Further, the ultrasound diagnostic apparatus 21 of the present embodiment can show dynamic blood flow in a state in which the luminance value is gradually reduced as time elapses, by performing processing based on the comparative table included in the lookup table 37d when showing dynamic blood flow during the decrease of blood flow velocity, in the blood flow image displayed on the monitor 41. Therefore, the ultrasound diagnostic apparatus 21 of the present embodiment can show dynamic blood flow more accurately than ever.

The present invention will not be limited by the above described embodiment; and it goes without saying that various modifications and applications can be made without deviating from the spirit of the invention.

What is claimed is:

1. An ultrasound diagnostic apparatus, comprising:
ultrasound transmitting/receiving unit for transmitting an ultrasound pulse to generate ultrasound to be transmitted from ultrasound transducing unit to a test subject, and receiving an echo signal outputted from the ultrasound transducing unit based on the ultrasound reflected in the test subject;
dynamic blood flow detection unit for calculating a predetermined detection value based on dynamic blood flow in the test subject based on the echo signal;
luminance value conversion unit for converting the predetermined detection value into a luminance value;
luminance value replacement unit retaining luminance value conversion information for converting one luminance value to another luminance value; and
comparison unit for comparing a first luminance value in one frame with a second luminance value in the frame immediately preceding the one frame, out of luminance values outputted from the luminance value conversion unit, wherein
the comparison unit is adapted to output either the first luminance value or a third luminance value, which is not larger than the absolute value of the second luminance value, based on the comparison result and the luminance value replacement information.

2. The ultrasound diagnostic apparatus according to claim 1, wherein the comparison unit is adapted to output the first luminance value when obtaining a comparison result that the first luminance value is larger than the second luminance value, and to replace the second luminance value with the third luminance value based on the luminance value replacement information, thereby outputting the third luminance value when obtaining a comparison result that the first luminance value is not larger than the second luminance value.

3. The ultrasound diagnostic apparatus according to claim 2, wherein the comparison unit is adapted to increase the attenuation of the third luminance value as the second luminance value increases when replacing the second luminance value with the third luminance value based on the luminance value replacement information.

4. The ultrasound diagnostic apparatus according to claim 2, wherein the luminance value replacement information retained by the luminance value replacement unit is configured to be a rewritable data file.

5. The ultrasound diagnostic apparatus according to claim 3, wherein the luminance value replacement information retained by the luminance value replacement unit is configured to be a rewritable data file.

6. The ultrasound diagnostic apparatus according to claim 4, wherein the data file is made up of a plurality of data files.

7. The ultrasound diagnostic apparatus according to claim 5, wherein the data file is made up of a plurality of data files.

8. The ultrasound diagnostic apparatus according to claim 1, wherein the comparison unit is adapted to output the first luminance value when obtaining a comparison result that the first luminance value has a different sign from that of the second luminance value, or that the first luminance value has the same sign as that of the second luminance value and the absolute value of the first luminance value is larger than that of the second luminance value; and to replace the second luminance value with the third luminance value based on the luminance value replacement information and output the third luminance value, when obtaining a comparison result that the first luminance value has the same sign as that of the second luminance and the absolute value of the first luminance value is not larger than that of the second luminance value.

9. The ultrasound diagnostic apparatus according to claim 8, wherein the comparison unit is adapted to increase the attenuation of the third luminance value as the second luminance value increases when replacing the second luminance value with the third luminance value based on the luminance value replacement information.

10. The ultrasound diagnostic apparatus according to claim 8, wherein the luminance value replacement information retained by the luminance value replacement unit is configured to be a rewritable data file.

11. The ultrasound diagnostic apparatus according to claim 9, wherein the luminance value replacement information retained by the luminance value replacement unit is configured to be a rewritable data file.

12. The ultrasound diagnostic apparatus according to claim 10, wherein the data file is made up of a plurality of data files.

13. The ultrasound diagnostic apparatus according to claim 11, wherein the data file is made up of a plurality of data files.

14. The ultrasound diagnostic apparatus according to claim 1, wherein the luminance value replacement information retained by the luminance value replacement unit is configured to be a rewritable data file.

15. The ultrasound diagnostic apparatus according to claim 14, wherein the data file is made up of a plurality of data files.

16. An ultrasound diagnostic apparatus, comprising:
ultrasound transmitting/receiving unit for transmitting an ultrasound pulse to generate ultrasound to be transmitted from ultrasound transducing unit to a test subject, and receiving an echo signal outputted from the ultrasound transducing unit based on the ultrasound reflected in the test subject;
dynamic blood flow detection unit for calculating a predetermined detection value based on dynamic blood flow in the test subject based on the echo signal;
luminance value conversion unit for converting the predetermined detection value into a luminance value;
storage unit for storing a first luminance value in one frame and a second luminance value in the frame immediately preceding the one frame, out of the luminance values and outputting the first luminance value and the second luminance value;
luminance value replacement unit for replacing the second luminance value outputted from the storage unit with a third luminance value which is not larger than the second luminance value to output the third luminance value; and
comparison unit for comparing the first luminance value outputted from the storage unit with the third luminance value outputted from the luminance value replacement unit to output one luminance value which is larger than the other luminance value.

17. The ultrasound diagnostic apparatus according to claim 16, wherein the comparison unit is adapted to increase the attenuation of the third luminance value as the second luminance value increases when replacing the second luminance value with the third luminance value based on the luminance value replacement information.

18. The ultrasound diagnostic apparatus according to claim 16, wherein the luminance value replacement information retained by the luminance value replacement unit is configured to be a rewritable data file.

19. The ultrasound diagnostic apparatus according to claim 17, wherein the luminance value replacement information retained by the luminance value replacement unit is configured to be a rewritable data file.

20. The ultrasound diagnostic apparatus according to claim 18, wherein the data file is made up of a plurality of data files.

21. The ultrasound diagnostic apparatus according to claim 19, wherein the data file is made up of a plurality of data files.

22. An ultrasound diagnostic apparatus, comprising:
ultrasound transmitting/receiving unit for transmitting an ultrasound pulse to generate ultrasound to be transmitted from ultrasound transducing unit to a test subject, and receiving an echo signal outputted from the ultrasound transducing unit based on the ultrasound reflected in the test subject;
dynamic blood flow detection unit for calculating a predetermined detection value based on dynamic blood flow in the test subject, based on the echo signal;
luminance value conversion unit for converting the predetermined detection value into a luminance value; and
comparison unit for comparing a first luminance value in one frame with a second luminance value in the frame immediately preceding the one frame, out of the luminance values outputted from the luminance value conversion unit, wherein
the ultrasound diagnostic apparatus further comprises a lookup table including a comparative table for converting the second luminance value into a third luminance value which is not larger than the absolute value of the second luminance value, and wherein
the comparison unit outputs either the first luminance value or the third luminance value based on the comparison result and the comparative table.

23. The ultrasound diagnostic apparatus according to claim 22, wherein the comparative table retained in the lookup table is configured to be a rewritable data file.

24. The ultrasound diagnostic apparatus according to claim 23, wherein the data file is made up of a plurality of data files.
